(19) United States
(12) Patent Application Publication
(10) Pub. No.: US 2005/0074900 A1
(43) Pub. Date: Apr. 7, 2005

(54) Microfluidic flow-through immunoassay for simultaneous detection of multiple proteins in a biological sample

(76) Inventors: Nicole Y. Morgan, Bethesda, MD (US); Michael Gaitan, North Potomac, MD (US); Laurie E. Locascio, North Potomac, MD (US); Terry M. Phillips, Washington, DC (US); Thomas J. Pohida, Monrovia, MD (US); Paul D. Smith, Annapolis, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/882,188

(22) Filed: Jul. 2, 2004

Related U.S. Application Data
(60) Provisional application No. 60/509,285, filed on Oct. 7, 2003.

(51) Int. Cl. G01N 33/558

(52) U.S. Cl. 436/514

(57) ABSTRACT

Described are microfluidic devices for, preferably, high-throughput, multi-analyte, affinity capture and detection of affinity-bindable analytes in biological fluids. Particularly, the devices can be used for immunoassays of biological fluids using multiple antibodies for capture and detection of multiple analytes, including proteins. The devices can be used for the simultaneous isolation and quantization of multiple proteins from microliter samples of biological fluids. Also described are methods for detecting and, optionally, quantifying, affinity-bindable analytes in biological fluids using these devices.

This application claims priority to U.S. Provisional Application No. 60/509,285 filed Oct. 7, 2003, the entire disclosure of which is incorporated herein by reference.

The invention includes microfluidic devices, preferably silicon chip-based, for, preferably, high-throughput, multi-analyte, affinity capture and detection of affinity-bindable analytes in biological fluids. Particularly, the devices can be used for immunoassays of biological fluids using multiple antibodies for capture and detection of multiple analytes, including proteins. The devices can be used for the simultaneous isolation and quantification of multiple proteins from microliter samples of biological fluids. Thus, the invention is also directed to methods for detecting and, optionally, quantifying, affinity-bindable analytes in biological fluids using the devices according to the invention.

Immunoassay tests are well-known in the art. They are designed to detect specific chemicals by measuring the chemicals' binding response to specific antibodies. Antibodies are known or will be developed which specifically bind with particular organic compounds, e.g., proteins. The antibodies do not respond to dissimilar substances. For example, in a simple immunoassay, antibodies may be coated inside a test tube. A sample is added to the test tube and proteins therein selectively bind to the antibodies. This can be followed by introducing a chemical that binds to and labels the proteins, e.g., a second antibody that is labeled with a fluorogenic or enzymatic reagent that can react to produce a color change. In this way a color change in an extracted solution can be connected with a specific protein presence or, optionally, quantifiable concentration.

The devices of the invention can be used in connection with known antibodies or those developed in the future for immunoassaying of known proteins. But the invention is not limited to antibody-protein assays. The devices of the invention can be used together with any type of surface chemistry which will selectively bind to a component of a biological fluid desired to be assayed. Advantages of the devices and methods of the invention include but are not limited to: the ability to run a single sample, in one run, through multiple sections having differing affinity binding surfaces so that the sample can be assayed for multiple analytes in a single experiment; the ability to assay small (e.g., sub-microliter) sample volumes; the ability to adjust the movement rate of a sample plug through differing sections; the ability to detect the presence of and, optionally, the quantity of, specifically bound analytes in the devices by direct observation or imaging; and, the ability to readily remove bound analytes from the devices so that the devices can be re-used for other samples.

The devices include a series of legs that are connected together so that fluid can be driven through each of them in series. But, at the same time, each leg can be addressed in parallel and independently. The basic idea for the operation of the device is to coat the surface of each of the legs, separately, with a film that will selectively and specifically bind to a compound (analyte) of interest. Preferably, the film coating in each leg will bind a different analyte. A sample with an unknown composition is sent down the entire device and each of the legs binds to and allows detection of the presence of their analyte of interest. In a preferred embodiment, an immunoassay approach for the surface coatings can be used. Another preferred embodiment is that the detection of the analyte is accomplished by an optical fluorescence measurement. The surface chemistry is preferably developed so that the device can be “reset” after each use, by removing the bound analytes, so that it is reusable for testing other samples.

The devices for the detection of two or more analytes in a biological fluid sample comprise:
a chip of a silicon material having open microchannels formed on one surface,
a wafer of a transparent material over the chip on the side with the open microchannels to enclose the microchannels, and
through-holes extending from the microchannels to the non-bonded side of the chip and/or wafer (i.e., to the exterior),
wherein the microchannels are arranged such that there is at least one main microchannel extending from an introduction port for the sample to an exit port for the sample and there are at least two microchannel legs, each microchannel leg having at each end a port, the ports being defined by a through-hole in the chip of silicon material or wafer material, and each microchannel leg crossing the main microchannel so that the volume of the microchannel leg and the main microchannel are co-extensive for some length, and
wherein at least one microchannel leg, on the inner surface of at least the portion where the volume of the microchannel leg and the main microchannel are co-extensive, has a coating of a binding material which specifically binds to an analyte and at least one other microchannel leg, on the inner surface of at least the portion where the volume of the microchannel leg and the main microchannel are co-extensive, has a coating of a binding material which specifically binds to a different analyte.

The chip of a silicon material having open microchannels formed on one surface and, optionally, through-holes from the other surface to the open microchannels can be of any silicon (or other) material which allows for formation of microchannels and through-holes therein and provides a surface chemistry amenable to coating with a material which specifically binds to an analyte. Silicon materials, particularly in chip form, are preferred which are used for photolithography patterning and etching methods to provide channels and through-holes therein. Preferably, after formation of the channels and through-holes, the silicon materials are surface treated to oxidize the surface to SiO₂ or add an SiO₂ layer thereon. This enhances the ability to modify the surface chemistry of the microchannels and permits electroosmotic pumping as discussed below. The silicon materials in chip form used are preferably of a size in the range of 2 mm to 15 cm long, 2 mm to 15 cm wide and 0.25 ml to 1 mm thick.

The microchannels are preferably in the range of about 10 to 100 μm deep and about 10 to 100 μm wide. The pattern of the microchannels is preferably of a serpentine pattern as exemplified in FIG. 1a, wherein a main channel zig-zags from one end of the chip to the other to provide an overall direction of flow of the main channel but which has a series of sections perpendicular to the main direction of flow wherein these sections intersect with and are co-extensive (i.e., they are the same channel at these sections) with the microchannel legs extending in that perpendicular direction. The microchannel legs continue to extend on both sides of the co-extensive section to introduction/exit ports outside of the main channel flow sections. For this purpose, the direction of flow in each successive microchannel leg is opposite to the one upstream. The ports are provided by the through-holes in the chip which are located to meet the ends of the channel legs and the sample inlet and outlet. This particular grid pattern is not required however and other patterns may be used as long as they provide a main channel and two or more crossing channel legs which have a portion co-extensive with the main channel. Preferably, the devices provide at least three up to about 100, more preferably 10 to 30, channel legs crossing and partially co-extensive with the main channel. The length of the portion of each channel leg which is co-extensive with the main channel can be chosen depending on the particular application and legs of differing length can be used in a single device. For example, this section length could be as low as 100 nm up to 10 cm, more preferably from 30 μm to 10 cm. The total length of the main channel, including the co-extensive and non-co-extensive sections, is preferably about 1 mm to 100 cm, more preferably about 110 cm to 100 cm.

The wafer of a transparent material over the chip is preferably of glass, more preferably a borosilicate glass, particularly a Pyrex™ glass, such as Corning 7740 glass. Other transparent materials may also be used such as transparent plastics including poly(dimethylsiloxane) (PDMS). The wafer of transparent material is provided over the surface of the silicon chip which contains the open microchannel pattern. This encloses the microchannels except for the through-hole ports which extend to the other side of the chip. The wafer is preferably of the same dimensions as the chip and about 100 μm to 2 mm thick.

The coating of a material which specifically binds to an analyte is provided on the inner surface of the microchannel at least in the section where the volume of the microchannel leg and the main microchannel are co-extensive. Preferably, a different coating, (each of which are specific to a different analyte) is provided in each channel leg. For these devices to be reusable, the binding materials should be strongly bound to the channel walls. The binding materials useful in the invention include many types of known binding materials which specifically bind to analytes desired to be detected in biological fluids. Any compounds which provide a specific molecular interaction with species of biological interest may be used. Particularly, materials already used for immunoassays may be used. Such binding materials include, but are not limited to: antibodies which specifically bind proteins; proteins which specifically bind antibodies; antibodies which specifically bind small organic molecules; antibodies which specifically bind cell surface antigens; DNA which specifically bind complementary DNA; RNA which specifically binds complementary RNA; aptamers which specifically bind small molecules; and aptamers which specifically bind proteins.

These binding materials must be attached to the microchannel surface. Surface chemistries known in the art for attaching such binding materials to a silicon material surface may be used. Particularly, the silicon chip surface is in an oxidized (Si—O⁻) form. When a glass wafer is used to form the top of the channel this also provides an Si—O⁻ surface chemistry. In this embodiment, a single surface chemistry can be used to coat all of these Si—O⁻ surfaces of the microchannel to the binding material. The surface preparation generally requires several treatment steps: for example, providing a reactive linking compound to the Si—O⁻ surface (often through a siloxane coupling chemistry), optionally reacting the linking compounds with a compound which will bind to the binding material, and then bonding the binding material to the linking compound or the intermediate compound. An advantage of the invention is that these treatment steps (or some of them) can be conducted separately on each of the microchannel legs to provide a different binding material in each. This is achieved through use of the ports at each end of the microchannel legs. The treatment solution is provided at an introduction port of the microchannel(s) of interest. Reservoirs at the ports can be provided for this purpose. The treatment solution is then driven through the microchannel leg to the exit port such that the surfaces of the microchannel are treated. The solution can be driven, for example, by application of a vacuum to the exit port, the use of a mechanical pump connected to the entrance port, or by electrical driving methods, as described below.

For illustration purposes, the following description is given of one type of surface chemistry treatment of the channel legs. First, the Si—O— surfaced channel walls of all the microchannel legs are functionalized by the attachment of a layer of aminosilane. To achieve this, the channels are filled with 1M NaOH for an hour at room temperature, rinsed with water, and then filled with 0.1M HCl for an hour. After a thorough rinsing with ultrapure water, the device is dried in a 125° C. oven for fifteen minutes and cooled for 3-5 minutes (relative humidity 65%, 22° C. throughout). All the channels are then filled with a 0.5% solution of 3-aminopropyl-triethoxysilane (APTES) in anhydrous toluene by covering all of the ports with the solution, placing the device in a dessicator, and repeatedly cycling between vacuum and atmospheric pressure in a toluene-saturated environment. The channels are left filled, with the chamber partially evacuated, for 15 hours. Afterwards, the device is rinsed with toluene, isopropanol, and ultrapure water, then dried and annealed at 125° C. for eighty minutes.

Then, streptavidin is covalently attached to the channel walls of all the legs. The channels are filled with a buffer containing an appropriate linker molecule, in this case a 5 mM solution of (Bis[sulfosuccinimidyl]suberate), BS³ (Pierce Chemical Co., Rockford, Ill.), in 20 mM HEPES, pH 7.4, and incubated for thirty minutes at room temperature. Then the channels are rinsed with buffer, filled with a 1 mg/ml solution of streptavidin, and incubated for an hour at room temperature and several days at 4° C.

Finally, the channels are rinsed thoroughly with fresh buffer, and differing biotinylated antibodies, which bind to streptavidin, are passed through each channel leg such that each leg has a different bound antibody; see, e.g., FIG. 1b. All channels are tilled with buffer, and the reservoirs for the unused legs closed off with a PDMS gasket and moderate pressure. The reservoir on one side of a selected channel leg is filled with a solution of a biotinylated antibody, which is drawn into the channel by applying vacuum to the other reservoir. After incubating for thirty minutes at room temperature, the channel is rinsed. This procedure is repeated with differing biotinylated antibodies for each channel leg.

The major advantage of using streptavidin-biotin chemistry in this example is the stability of the streptavidin coating. The streptavidin can be covalently linked to all of the channel walls well before the antibodies are attached, and the attachment of the biotinylated antibody itself is a single-step process. Additionally, the streptavidin coating helps reduce the problem of nonspecific binding of antibodies and sample proteins. However, other surface chemistries can be used according to the invention.

The devices of the invention are useful in methods for the detection and, optionally, quantification of two or more analytes in a biological fluid sample. The biological fluids contemplated for assay by the devices include, particularly blood and other bodily fluids including cerebrospinal fluid, plasma, urine, tears, saliva, mucous, cervical secretions, and wound discharges. The fluids are preferably used untreated but may be centrifuged and/or filtered, if necessary. The analytes for detection can be any that can be specifically bound by the surface chemistry provided on the microchannel surface. Preferred embodiments provide surface chemistry for specific binding of proteins, antibodies, antigens, toxins, complementary DNA, complementary RNA and aptamers. In one embodiment, the device can be used to assay for proteins related to the onset of cancer or particular cancer types. Tests with the devices can be conducted on an array of proteins to see if some or all are predictive of the cancer.

The analytes must be tagged or labeled in a manner which allows their detection after binding. Methods for such tagging or labeling of particular types of analytes and subsequent detection are known in the art and any of these methods, or newly developed methods therefore, can be used. Particularly useful labeling methods include fluorescent labeling of the analyte, or enzyme labeling of the analyte through a second antibody. For example, the labeling step can be performed prior to or simultaneously with the analyte binding step (in the first case of fluorescent labeling) or it can be performed after the sample is flowed through the device (as in the second case of enzyme labeling).

An advantage of the invention is that small volume samples, e.g., from less than 1 nl to 100 μl, more preferably about 0.1 μl can be assayed. The samples are introduced into the device through the introduction port of the main channel and then driven to the exit port of the main channel. This can be done by applying a vacuum to the exit port and blocking all of the channel leg ports. But, preferably, it is done by electrically driving a sample plug through the main microchannel of the device.

For controlling the flow of the sample through the device; for example, as in FIG. 1c, electrical control of the flow is preferred. When the solution in the device contains a salt and the wall surface is provided with a charge, electro-osmotic flow can be induced by applying a potential difference between two points to provide flow from high to low potential. Two main issues had to be addressed in using this electrokinetic flow control in these silicon/glass devices.

First, biological buffers typically contain large amounts of sodium ions, which are fairly mobile in silicon dioxide. As a result, the silicon wafer must be maintained at either positive or zero voltage with respect to all portions of the channel. If the wafer is negative with respect to the channel, sodium ions from the buffer will be drawn into the oxide layer, leading to electrical breakdown.

Second, the breakdown voltage of the oxide limits the voltage that can be applied along the channel. The full length of, for example, a device with twenty channel legs may be approximately 30 cm. Typical linear flow rates of a few cm/min require fields on the order of 100 V/cm, which translates into a voltage of 3 kV if applied across the entire device. However, the anodic bonding process used to attach the glass wafer (described below) fails if the oxide is much thicker than 7000 Å. In order to stay well within typical oxide breakdown fields of 106 V/cm, the voltages applied to the channel reservoirs should not be more than around 500 V.

To overcome this difficulty, a set of computer-controlled high-voltage relays can be used, which allow applying a voltage difference, up to 500V, across only the section of the channel that contains the sample plug. As the sample moves along the channel, more of the reservoirs are switched from ground to high voltage, permitting reasonable flow rates through a 30-cm device with sub-kilovolt applied voltages. Computerized control of the sample flow also permits the residence time of the sample plug in each channel leg to be adjusted independently, in order to optimize binding to the immobilized binding material (e.g., antibodies). Preferably, a potential is applied to achieve a flow rate of the sample plug through the main microchannel of a few cm/min, e.g., 0.5-5 cm/min.

After the sample has been driven through all of the channel legs, the labeled analytes of interest therein will be bound to their respective binding materials in the respective channel legs. The labeled analytes can now be detected and, optionally, quantified. The method of detecting the labeled analytes will depend on the manner of labeling and known methods for detection of the labeled analytes can be used. The wafer of transparent material allows for optical detection and, optionally, quantification by fluorescent or chemiluminescent labels. For example, for fluorophore-labeled materials, the chip can be exposed to a laser to excite the fluorophores and then the location and intensity of the fluorescence can be determined, e.g., using scanning and CCD imaging techniques. Location of a labeled analyte at a particular channel leg will indicate that the sample contained an analyte specifically bound by the binding material in that particular channel leg. The intensity of the fluorescence will be indicative of the quantity of that analyte in the sample. Because the channel legs have a material which specifically binds to different analytes, the devices advantageously allow assessment of the presence and amount of multiple analytes of interest in a single sample assay run.

For the manipulation of aqueous buffers and provision of reservoirs for the above purposes, a strip of PDMS can be placed over the ports. Holes cut in the PDMS can serve as fluid reservoirs, and confine the liquid to individual ports. In order to thoroughly rinse all of the channels without introducing air bubbles, a preferred procedure outlined in FIG. 3 was developed. The reservoirs in three rows are filled as indicated, and vacuum applied to the reservoirs in the fourth row until approximately 2 μl of flow is observed. After these wells are filled, the two end reservoirs are emptied, vacuum applied, and the reservoirs refilled.

The devices according to the invention can be made using techniques known in the art but adapted to achieve the novel design of the invention. For example, the main microchannel and microchannel legs may be fabricated in the silicon chip with transparent wafer, e.g., glass, cover using a two-step photolithography process in combination with an anisotropic wet-chemical etch (TMAH), followed by anodic bonding. A cross-section of the construct is shown in FIG. 2. In the first photolithographic step, the through-holes (not shown) for fluidic access to the device are patterned in the back of the silicon chip. In the second step, the serpentine channel pattern (as in FIG. 1a) is formed on the front of the chip. After the patterning is complete, the silicon chips are cleaned, and a thick (6600 Å) wet oxide is grown on the surface for electrical isolation. Finally, the channels are sealed using an anodic bonding technique: for example, a 0.5 mm thick glass wafer (Corning 7740) is brought into contact with the front of the silicon chip, and the silicon/glass sandwich is heated to 400° C. with an applied voltage of 1200V. Modifications may be made to these photolithography, etching and anodic bonding methods according to the knowledge in these arts.

Among the advantages of this structure are the following. First, putting the through-holes on the back of the silicon chip permits full optical access to the channels through the glass wafer on the top of the silicon chip, making more efficient light collection possible. Second, the side ports on each channel (see FIG. 1) permit separate addressing of each channel leg, as described above. Third, the use of a silicon substrate makes it easier to incorporate additional on-chip functionality. Fourth, the surface chemistry for attachment of biomolecules to glass/silicon dioxide is well developed and can be used for providing differing binding material on the microchannel legs. Finally, the anodic bonding technique provides a robust and reproducible seal for the channels; as the entire device occupies a large wafer area, for example, about 3 cm×10 cm, a high device yield would not otherwise be feasible.

The entire disclosure of all applications, patents and publications, cited herein and of U.S. provisional application No. 60/509,285, filed Oct. 7, 2003, is incorporated by reference herein.

### BRIEF DESCRIPTION OF THE DRAWINGS

FIG. 1 is a schematic of one embodiment of device operation showing: a) an overall device structure; b) antibody loading into individual legs; and c) sample analysis.

FIG. 2 is a cross-section of an anodically bonded silicon/glass channel according to an embodiment of the invention, together with schematic of a method for attachment of antibodies and detection of captured proteins. In this embodiment, all surfaces of the channel leg are chemically similar, and so the antibodies will be attached to all four walls.

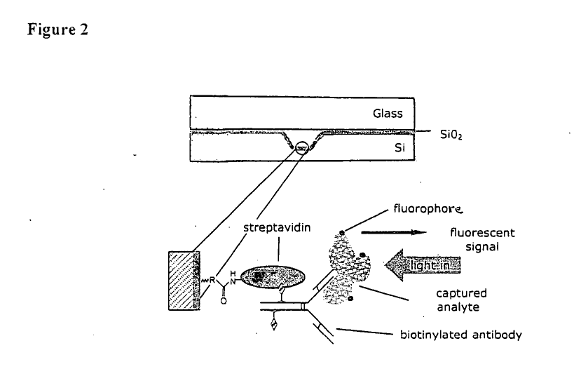

FIG. 3 is a schematic of one embodiment for filling and rinsing of the channel legs of a device according to the invention.

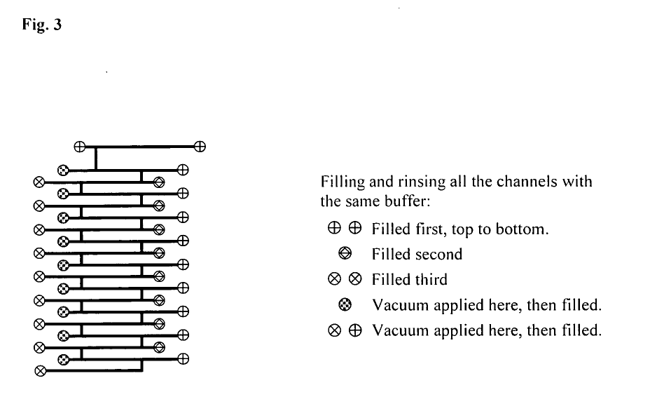

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### Example 1

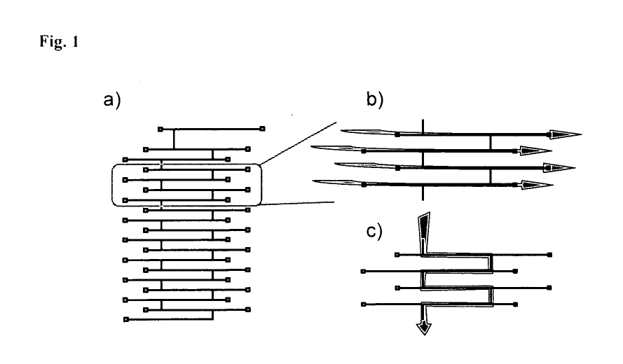

In a preferred embodiment of the invention, the device consists of a long glass-coated channel, 50 μm wide×15 μm deep×30 cm long, with a serpentine pattern etched in a silicon chip; for example, as shown in FIG. 1a. Side ports on each of the channel legs are used for pressure-driven or electrically driven loading of different biotinylated antibodies into each segment of the channel legs, independently; see, e.g., FIG. 1b. These antibodies bind to streptavidin that has already been covalently linked to the channel surfaces. After the antibodies have been immobilized, all of the proteins in the sample are tagged with fluorescent molecules and the sample is flowed through the main microchannel of the device, as exemplified by the directional arrow in FIG. 1c. After rinsing, laser-induced fluorescence is used to detect the amount of protein bound in each channel leg. By flowing an acidic pH gradient through the device, it is then possible to disrupt the antibody-antigen binding, thereby removing the bound sample proteins. The device, with the bound antibodies intact, can then be reused for additional samples; furthermore, this reusability permits the device to be calibrated before the first sample is measured, allowing more accurate quantitative measurements of the sample proteins. This device architecture has several advantages over existing array technology. For example: the proteins can be detected by single-point capture, and much smaller sample volumes can be used. In addition, it is preferred to be able to reuse the channels with the bound antibodies for multiple samples, greatly reducing the cost of analyzing each sample. This device and others according to the invention can be integrated into other analytic equipment or on-chip detectors.

The preceding examples can be repeated with similar success by substituting the generically or specifically described reactants and/or operating conditions of this invention for those used in the preceding examples.

From the foregoing description, one skilled in the alt can easily ascertain the essential characteristics of this invention and, without departing from the spirit and scope thereof, can make various changes and modifications of the invention to adapt it to various usages and conditions.

1. A device for the detection of two or more analytes in a biological fluid sample which device comprises:
a chip of a silicon material having open microchannels formed on one surface,
a wafer of a transparent material bonded over the chip on the side with the open microchannels to enclose the microchannels, and
through-holes extending from the microchannels to the non-bonded side of the chip and/or wafer,
wherein the microchannels are arranged such that there is at least one main microchannel extending from an introduction port for the sample to an exit port for the sample and there are at least two microchannel legs, each microchannel leg having at each end a port, the ports being defined by a through-hole in the chip of silicon material or in the wafer, and each microchannel leg crossing the main microchannel so that the volume of the microchannel leg and the main microchannel are co-extensive for some length, and
wherein at least one microchannel leg, on the inner surface of at least the portion where the volume of the microchannel leg and the main microchannel are co-extensive, has a coating of a binding material which specifically binds to an analyte and at least one other microchannel leg, on the inner surface of at least the portion where the volume of the microchannel leg and the main microchannel are co-extensive, has a coating of a binding material which specifically binds to a different analyte.

2. The device of claim 1, wherein the silicon chip and wafer of transparent material are of the same width and length being 2 mm to 15 cm long and 2 mm to 15 cm wide and have, together, a thickness of 0.25 μm to 5 mm.

3. The device of claim 1, wherein the wafer of transparent material is of a borosilicate glass.

4. The device of claim 1, wherein the silicon chip is oxidized on its surface contacting the wafer of transparent material and the surfaces of the through-holes, the main microchannel and the microchannel legs.

5. The device of claim 1, wherein the main microchannel and microchannel legs are in the range of about 10 to 100 μm deep and about 10 to 100 μm wide.

6. The device of claim 1, wherein the microchannels are arranged in a serpentine pattern such that the main microchannel sections which are not co-extensive with the microchannel legs are perpendicular to the microchannel legs.

7. The device of claim 1, which contains 10-30 microchannel legs.

8. The device of claim 1, wherein the length of the portion of each microchannel leg which is co-extensive with the main microchannel is from 30 nm to 10 cm.

9. The device of claim 1, wherein the total length of the main microchannel, including the co-extensive and non-co-extensive sections, is about 10 to 100 cm.

10. The device of claim 1, wherein a different coating of binding material, each of which is specific to a different analyte, is provided in each microchannel leg.

11. The device of claim 1, wherein the binding materials are antibodies which specifically bind proteins, proteins which specifically bind antibodies, antibodies which specifically bind small organic molecules, antibodies which specifically bind cell surface antigens, DNA which specifically bind complementary DNA, RNA that which specifically binds complementary RNA, aptamers which specifically bind small molecules, or aptamers which specifically bind proteins.

12. The device of claim 1, wherein the binding materials are antibodies which specifically bind proteins.

13. The device of claim 12, wherein the antibodies are bound to the microchannel legs through a linking group bound to Si—O⁻ on the microchannel surface, which is bound to streptavidin which is bound to a biotinylated form of the antibody.

14. The device of claim 1, wherein the device is arranged with electrodes located to be capable of driving a sample plug through the main microchannel when voltage is applied.

15. The device of claim 14, wherein the electrodes are located to also be capable of driving solutions through each microchannel leg from the port at one end to the port at the other end.

16. The device of claim 14, wherein the electrodes are located to be capable of driving a sample plug at differing rates for each section of the main microchannel which is co-extensive with the microchannel legs.

17. The device of claim 14, wherein electrodes spaced along the main channel, together with high-voltage relays, allows the rapid electrical movement of a few cm/min of a sample plug along a 10-100 cm channel without exceeding an applied voltage of 500V.

18. A method for assaying a biological fluid sample for two or more analytes, which comprises:
treating the biological fluid sample to tag analytes therein with a tag allowing its detection,
passing the sample through the main microchannel of a device according to claim 1,
detecting and, optionally, quantifying the tagged analytes bound to the binding material in the co-extensive section of each microchannel leg of the device.

19. A method for assaying a biological fluid sample for two or more analytes, which comprises:
passing the sample through the main microchannel of the device according to claim 1,
passing a reagent through the microchannel that can further bind to the bound sample to tag it,
detecting and, optionally, quantifying the tagged analytes bound to the binding material in the co-extensive section of each microchannel leg of the device.

20. The method of claim 18, wherein the sample is passed through the main microchannel as a sample plug driven by use of electrical potential.

21. The method of claim 20, wherein the sample plug is driven by sequential application of potential along differing sections of the main microchannel.

22. The method of claim 18, wherein the sample plug is driven by mechanically driven flow.

23. The method of claim 18, wherein the sample is a blood sample.

24. The method of claim 18, wherein the binding materials in the microchannel legs are antibodies which specifically bind proteins in the sample, proteins which specifically bind antibodies in the sample, antibodies which specifically bind small organic molecules in the sample, antibodies which specifically bind cell surface antigens in the sample, DNA which specifically bind complementary DNA in the sample, RNA that which specifically binds complementary RNA in the sample, aptamers which specifically bind small molecules in the sample, or aptamers which specifically bind proteins in the sample.

25. The method of claim 18, wherein the binding materials in the microchannel legs are antibodies which differ in each microchannel leg and the antibodies specifically bind protein analytes in the sample.

26. The method of claim 18, wherein the device has 10-30 microchannel legs each of which specifically bind to a different analyte.

27. The method of claim 18, wherein the analytes are tagged with a fluorophore and the tagged analytes bound in the device are detected and, optionally, quantified by observation or imaging of the fluorophorescence through the transparent material.

28. The method of claim 25, wherein the protein analytes are tagged with a fluorophore, after passing the sample through the device, the device is subject to a laser to activate the fluorophores, a CCD image is taken of the device and the image analyzed for location and intensity of fluorescence to detect and quantify the specific proteins bound by the antibodies.

29. The method of claim 18, wherein the sample volume is about 1 μl.

30. The method of claim 18, which further comprises, after detection of the tagged analytes, passing a solution of acidic pH through the main microchannel to remove the bound analytes and passing another sample through the device.

31. A method for preparing a device according to claim 1, which comprises at least one step of separately treating each microchannel leg to provide the coating of a binding material which specifically binds to an analyte distinct from binding material in other microchannel legs, by passing at least one treatment solution from one end port to the other end port of each microchannel leg separately.
